(19) United States
(12) Patent Application Publication
(10) Pub. No.: US 2025/0188410 A1
(43) Pub. Date: Jun. 12, 2025

(54) NICOTINAMIDE ADENINE DINUCLEOTIDE (NAD+) PRECURSORS, NAD PATHWAY SUBSTRATES COMPOSITIONS AND USES THEREOF

(71) Applicant: Colossal Biosciences Inc., Austin, TX (US)

(72) Inventors: Eriona Hysolli, Northfield, NH (US); Mariana Ianello Giassetti, Georgetown, TX (US)

(21) Appl. No.: 18/977,882

(22) Filed: Dec. 11, 2024

Related U.S. Application Data
(60) Provisional application No. 63/608,613, filed on Dec. 11, 2023.

(51) Int. Cl. C12N 5/073

(52) U.S. Cl. CPC C12N 5/0603; C12N 2500/32; C12N 2500/40; C12N 2500/42; C12N 2500/46; C12N 2506/02

(57) ABSTRACT

This disclosure is directed to culture media containing a nicotinamide adenine nucleotide (NAD⁺) precursor and/or a NAD pathway substrate. This disclosure is also directed to in vitro methods of improving the cell state in an embryo by contacting a mammalian embryo with a culture medium supplemented with a NAD⁺ precursor and/or a NAD pathway substrate. The improvements to the cell state may include increasing the presence of an expanded blastocyst, increasing the attachment rate of the embryo, and/or increasing the derivation of embryonic stem cells from the embryo. In certain embodiments, the media and methods use a NAD⁺ precursor. A variety of different NAD⁺ precursors can be used in the media and methods, including nicotinic acid (NA) and nicotinamide mononucleotide (NMN).

### CROSS-REFERENCE TO RELATED APPLICATIONS

This application claims priority to U.S. Provisional Application 63/608,613 (filed on Dec. 11, 2023) which is incorporated by reference in its entirety.

### FIELD OF THE INVENTION

This disclosure relates to culture media containing nicotinamide adenine nucleotide (NAD⁺) precursors and/or NAD pathway substrates and uses of such NAD⁺ precursors and/or NAD pathway substrates to improve the cell state in mammalian embryos.

### BACKGROUND OF THE INVENTION

NAD⁺ precursors, such as Nicotinamide mononucleotide (NMN) and Nicotinic Acid (NA, also known as Niacin), showed improvement in some age-related disease mouse models, leading to ongoing clinical trials to assess their safety and benefits (Miao Y et al., Cell Rep 2020; 32:107987). The NAD⁺ precursors NA and NMN had fewer reported unfavorable side effects.

Understanding NAD⁺ production and consumption pathways is crucial for precise insights and therapies targeting age-related conditions such as female infertility (Miao Y et al., Cell Rep 2020; 32:107987). NAD⁺ synthesis is indispensable for mice oocyte and pig oocyte developmental competence as well as their pre-implantation development. Moreover, NAD⁺ precursors rescue premature ovarian failure and improve the early embryonic development of aged oocytes when administered in vivo or supplemented in vitro during the oocyte maturation in pigs, cows, and mice (Miao Y et al., Cell Rep 2020, 32:107987; Hashimoto S et al., 38th Hybrid Annu Meet ESHRE, 2022, 2019:3-4; Pollard C L et al., Reprod Domest Anim 2022, 68:345-354; Pollard C L et al., J Reprod Dev 2021, 67:319-326). In vitro supplementation of NA during early pig embryo development has been tested (Almubarak A et al., Reprod Domest Anim 2023, 58:1685-1694).

Hashimoto et al. observed that addition of nicotinamide adenine dinucleotide (NAD1) precursor to oocyte maturation medium improves the developmental competence of bovine oocytes after IVF (Hashimoto S et al., 38th Hybrid Annu Meet ESHRE, 2022, 2019:3-4). Nicotinamide adenine dinucleotide has also been shown to induce a bivalent metabolism and maintain pluripotency in human embryonic stem cells (Lees J. et al., Stem Cells 2020, 38:624-638). Furthermore, it has been “demonstrated that oxidized nicotinamide adenine dinucleotide (NAD⁺) synthesis is indispensable for mouse embryo pre-implantation development.” Li, J. et al., Cell Discovery 2022, 8:96.

In contrast, Pollard et al. observed that “[s]upplementing the IVM [in vitro maturation] or embryo culture media with NMN had no effect on maturation or embryo development.” Pollard C L et al., J. Reprod. Dev. 2021, 67:319-326. Thus, in vitro effects of NAD⁺ precursors on embryonic development, micromanipulated embryos/oocytes survival rate, and efficacy of in vitro embryonic stem cells (ESCs) derivation are mainly unknown.

### SUMMARY OF THE INVENTION

The disclosure provides in vitro methods of improving the cell state in an embryo (e.g., increasing the presence of an expanded blastocyst, increasing the attachment rate of the embryo, and/or increasing the derivation of embryonic stem cells from the embryo). The disclosure also provides culture media for improving the cell state in an embryo.

One embodiment of the disclosure is an in vitro method of improving the cell state in an embryo that includes contacting a mammalian embryo with a culture medium supplemented with a nicotinamide adenine dinucleotide (NAD⁺) precursor and/or a NAD pathway substrate. In certain embodiments, the contacting includes culturing the mammalian embryo for 3 to 14 days, alternatively for at least 3 days, alternatively for at least the first seven days of embryo development.

In one embodiment, the contacting includes culturing the mammalian embryo for at least 3 days, followed by further includes culturing the mammalian embryo in a culture medium for at least 7 days, optionally the contacting further includes transferring the embryo to a culture medium for a further at least 7 days. In some embodiments, the contacting includes culturing the mammalian embryo on a feeder layer. In certain embodiments, when the mammalian embryo is cultured on a feeder layer, the culture medium is supplemented with a rho-associated protein kinase (ROCK) inhibitor for the last twelve hours of culturing. In other embodiment, a ROCK inhibitor is used after passaging.

In certain embodiments, the culture medium is supplemented with a NAD⁺ precursor. In other embodiments, the culture medium is supplemented with a NAD pathway substrate. In other embodiments, the culture medium is supplemented with a NAD⁺ precursor and a NAD pathway substrate.

In certain embodiments, culture medium used in the method is supplemented with from about 125 μM to about 1000 μM, alternatively from about 250 μM to about 750 μM, or about 500 μM of the NAD⁺ precursor and/or the NAD pathway substrate.

In some embodiments, the improving the cell state includes increasing the presence of an expanded blastocyst, increasing the attachment rate of the embryo, and/or increasing the derivation of embryonic stem cells from the embryo. In one embodiment, the improving the cell state includes increasing the presence of an expanded blastocyst. In another embodiment, the improving the cell state includes increasing the attachment rate of the embryo. In yet another embodiment, the improving the cell state includes increasing the derivation of embryonic stem cells from the embryo.

The mammalian embryo used in the method can be obtained by a variety of ways. In one embodiment, the mammalian embryo was obtained by in vitro fertilization (IVF). In another embodiment, the mammalian embryo was obtained by parthenogenetic activation (PA). In an alternate embodiment, the mammalian embryo was obtained by interspecies somatic cell nuclear transfer, somatic cell nuclear transfer, intracytoplasmic sperm injection, and embryonic complementation. In certain embodiments, the method increases the attachment rate of PA embryos. In other embodiments, the method increases the derivation of embryonic stem cells from IVF and PA embryos. In additional embodiments, the method increases the derivation of embryonic stem cells from SCN/iSCNT embryos.

A variety of NAD⁺ precursors can be used. For example, the NAD⁺ precursor is selected from the group consisting of tryptophan, nicotinic acid (pyridine-3-carboxylic acid) (NA), nicotinamide (nicotinic acid amide), nicotinamide mononucleotide (NMN), nicotinamide riboside, and combinations thereof. In one embodiment, the NAD⁺ precursor is NA and/or NMN. In another embodiment, the NAD⁺ precursor is NMN.

A variety of different NAD pathway substrates can be used. Exemplary suitable NAD pathway substrates include but are not limited to ethanol, lactate, glyceraldehyde-3-phosphate, malate, and glutamate. In certain embodiments, the NAD pathway substrate is selected from the group consisting of lactate, glyceraldehyde-3-phosphate, malate, glutamate, and combinations thereof.

Another embodiment of the disclosure is a culture medium for improving the cell state in an embryo containing: a basic culture medium; and an effective amount of a NAD⁺ precursor and/or a NAD pathway substrate to improve the state of cells in a mammalian embryo. The improving the cell state includes increasing the presence of expanded blastocyst, increasing the attachment rate of embryos, and/or increasing the derivation of embryonic stem cells from the embryo. The improving can also include in vitro embryo production.

In certain embodiments, the culture medium contains a basic culture medium and an effective amount of a NAD⁺ precursor. In one embodiment, the culture medium contains from about 125 μM to about 1000 μM of the NAD⁺ precursor. In another embodiment, the NAD⁺ precursor is selected from the group consisting of tryptophan, NA, nicotinamide, NMN, nicotinamide riboside, and combinations thereof. In an alternate embodiment, the NAD⁺ precursor is NMN, NA, or a combination thereof. In one embodiment, the basic culture medium is BO-IVC, bEPSCM, or mTeSR.

In other embodiments, the basic culture medium is: (a) DMEM/F12 supplemented with bFGF, LiCl, GABA, pipecolic acid and TGFß; or (b) DMEM/F12 supplemented with bFGF, LiCl, TGFß, and optionally GABA, pipecolic acid, or combinations thereof. In further embodiments, the basic culture medium is supplemented with bFGF, LiCl, GABA, pipecolic acid and TGFß. In additional embodiments, the basic culture medium is supplemented with bFGF, LiCl, TGFß, and optionally GABA, pipecolic acid, or combinations thereof. In certain embodiments, the basic culture medium is further supplemented with XAV939, CHIR99021, WH-4-023, A419259, Vitamin C, Activin A, and LIF. In another embodiment, the basic culture medium is further supplemented with IWR-1, CHIR99021, WH-4-023, A419259, Vitamin C, Activin A, and LIF.

In other embodiments, the culture medium contains a basic culture medium and an effective amount of a NAD pathway substrate. In one embodiment, the culture medium contains from about 125 μM to about 1000 μM of the NAD pathway substrate. In certain embodiments, the NAD pathway substrate is selected from the group consisting of lactate, glyceraldehyde-3-phosphate, malate, glutamate, and combinations thereof.

Other features and advantages of the invention will be apparent from the detailed description and examples that follow.

### BRIEF DESCRIPTION OF THE DRAWINGS

The foregoing summary, as well as the following detailed description of the invention, will be better understood when read in conjunction with the appended figures. For the purpose of illustrating the invention, the figures demonstrate embodiments of the present invention. It should be understood, however, that the invention is not limited to the precise arrangements, examples, and instrumentalities shown.

FIG. 1 shows the effects of NMN treatment on the cleavage rate on day 3 (D3), the blastocyst rate on day 7 (D7), and the hatching rate on day 11 (D11) on bovine embryos as a function of embryonic development stage.

FIG. 2A shows the effects of NMN treatment on hatched blastocyst morphology on day 11 (D11). FIG. 2B shows the effects of NMN treatment on hatched blastocyst morphology on day 11-12 (D11-12).

FIG. 3 shows the effects of NMN treatment on the cleavage rate on day 3 (D3), the morula rate on day 5 (D5), the blastocyst rate on day 7 (D7), and the hatching rate on Day 11 (D11) on bovine embryos as a function of embryonic development stage.

FIG. 4 shows representative image of embryo development at day 7 (D7) and day 12 (D12) after supplementation of NAD⁺ precursor; the black arrows indicate compact morphology, and the white arrows indicate expanded hatched blastocysts. The center panel of FIG. 4 shows treatment with 1 mM of NA. The right hand panel of FIG. 4 shows treatment with 0.5 mg/ml of NMN.

FIG. 5 shows a representative image of measurements of attached embryos at day 14 (D14) (IVF, no NMN treatment).

FIG. 6 shows a representative image of staining an attached embryo for Alkaline Phosphatase.

### DETAILED DESCRIPTION

This disclosure is based on the discovery that treating mammalian embryos with a NAD⁺ precursor and/or a NAD pathway substrate improves the cell state in the embryo, such e.g., improved functionality and efficiency of embryo development. In particular, this disclosure is based on the discovery that contacting a mammalian embryo with a NAD⁺ precursor for a sufficient time, such as e.g., 3 days to 14 days, increases the presence of an expanded blastocyst, increases the attachment rate of the embryos, and/or increases the derivation of embryonic stem cells from the embryo.

The general description and the following detailed description are exemplary and explanatory only and are not restrictive of the invention, as defined in the appended claims. Other aspects of the present invention will be apparent to those skilled in the art in view of the detailed description of the invention as provided herein.

For clarity of disclosure, and not by way of limitation, the detailed description of the invention is divided into subsections that describe or illustrate certain features, embodiments, or applications of the present invention.

### Definitions

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods, and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates, which may need to be independently confirmed.

It is noted that, as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

As used herein, the term “about” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

It is also to be understood that the terminology used herein is for the purpose of describing particular embodiments only and is not intended to be limiting.

As used herein, the terms “comprising,” “including,” “containing” and “characterized by” are exchangeable, inclusive, open-ended and do not exclude additional, unrecited elements or method steps. Any recitation herein of the term “comprising,” particularly in a description of components of a composition or in a description of elements of a device, is understood to encompass those compositions and methods consisting essentially of and consisting of the recited components or elements.

As used herein, the term “consisting of” excludes any element, step, or ingredient not specified in the claim element.

It should also be understood that the terms “about,” “approximately,” “generally,” “substantially” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

Before certain embodiments are described in greater detail, it is to be understood that this invention is not limited to certain embodiments described, as such may, of course, vary. It is also to be understood that the terminology used herein is for the purpose of describing certain embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

It is also to be understood that the terminology used herein is for the purpose of describing particular embodiments only and is not intended to be limiting.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “stem cell” refers to a cell that can self-renew and differentiate to at least one more-differentiated or less developmentally-capable phenotype. The term “stem cell” encompasses stem cell lines, induced stem cells, non-human embryonic stem cells, pluripotent stem cells, multipotent stem cells, amniotic stem cells, placental stem cells, or adult stem cells. An “induced stem cell” is one derived from a non-pluripotent cell induced to a less-differentiated or more developmentally-capable phenotype by introduction of one or more reprogramming factors or genes. As the term is used herein, an induced stem cell need not be pluripotent, but has the capacity to differentiate, under appropriate conditions, to more than one more-highly-differentiated phenotype. It should be understood that the capacity was not present prior to the introduction of reprogramming factors. An induced stem cell will express at least one stem cell marker not expressed by the parent cell prior to introduction of reprogramming factors. In this context, a stem cell marker is exclusive of a factor introduced by reprogramming. An induced pluripotent stem cell, or iPS cell, has the induced capacity to differentiate, under appropriate conditions, to a cell phenotype derived from each of the endoderm, mesoderm, and ectoderm germ layers.

As used herein, the term “somatic cell” refers to any cell other than a germ cell, a cell present in or obtained from a pre-implantation embryo, or a cell resulting from proliferation of such a cell in vitro. Stated another way, a somatic cell refers to any cells forming the body of an organism, excluding germ cells. Every cell type in the mammalian body-apart from the sperm and ova and the cells from which they are made (gametocytes)—is a somatic cell: internal organs, skin, bones, blood, and connective tissue are all substantially made up of somatic cells. In some embodiments the somatic cell is a “non-embryonic somatic cell,” by which is meant a somatic cell that is not present in or obtained from an embryo and does not result from proliferation of such a cell in vitro. In some embodiments, the somatic cell is an “adult somatic cell,” by which is meant a cell that is present in or obtained from an organism other than an embryo or a fetus or results from proliferation of such a cell in vitro.

As used herein, the phrase “improving the cell state in an embryo” refers to the beneficial effects that result from treatment of a mammalian embryo with a NAD⁺ precursor and/or a NAD pathway substrate. These beneficial effects include but are not limited to increasing the presence of an expanded blastocyst, increasing the attachment rate of embryos, and increasing the derivation of embryonic stem cells from the embryo. The improving can also include in vitro embryo production. In certain embodiments, the phrase “improving the cell state in an embryo” refers to increasing the presence of an expanded blastocyst, increasing the attachment rate of embryos, and/or increasing the derivation of embryonic stem cells from the embryo as a result of treating a mammalian embryo with a NAD⁺ precursor.

It is also to be understood that the terminology used herein is for the purpose of describing particular embodiments only and is not intended to be limiting.

Before certain embodiments are described in greater detail, it is to be understood that this invention is not limited to certain embodiments described, as such may, of course, vary. It is also to be understood that the terminology used herein is for the purpose of describing certain embodiments only, and is not intended to be limiting, since the scope of the present invention will be limited only by the appended claims.

NAD⁺ Precursor and/or NAD Pathway Substrate Containing Culture Media

One aspect of the disclosure is directed to a NAD⁺ precursor and/or a NAD pathway substrate containing culture media. In particular, the disclosure provides culture media for improving the cell state in an embryo. The culture media contain a basic culture medium and an effective amount of a NAD⁺ precursor and/or a NAD pathway substrate to improve the state of cells in an embryo, such as e.g., a mammalian embryo.

In one embodiment, the culture medium contains a basic culture medium and an effective amount of a NAD⁺ precursor. In another embodiment, the culture medium contains a basic culture medium and an effective amount of a NAD pathway substrate.

In one embodiment, the improving the cell state includes increasing the presence of expanded blastocysts, increasing the attachment rate of embryos, and/or increasing the derivation of embryonic stem cells from the embryo.

In certain embodiments, the effective amount of a NAD⁺ precursor in the culture medium is from about 125 μM to about 1000 μM, alternatively from about 150 μM to about 950 μM, alternatively from about 125 μM to about 900 μM, alternatively from about 200 μM to about 800 μM, alternatively from about 250 μM to about 750 μM, alternatively from about 225 μM to about 700 μM, alternatively from about 250 μM to about 600 μM, alternatively from about 300 μM to about 600 μM, alternatively from about 350 μM to about 600 μM, alternatively from about 400 μM to about 600 μM, alternatively about 500 μM.

A variety of different NAD⁺ precursors can be used. In one embodiment, the NAD⁺ precursor is selected from the group consisting of tryptophan, nicotinic acid (pyridine-3-carboxylic acid) (NA), nicotinamide (nicotinic acid amide), nicotinamide mononucleotide (NMN), nicotinamide riboside, and combinations thereof. In another embodiment, the NAD⁺ precursor is selected from the group consisting of nicotinic acid (pyridine-3-carboxylic acid) (NA), nicotinamide (nicotinic acid amide), nicotinamide mononucleotide (NMN), nicotinamide riboside, and combinations thereof. In another embodiment, the NAD⁺ precursor is NA, NMN, and a combination thereof. In yet another embodiment, the NAD⁺ precursor is NA. In an alternate embodiment, the NAD⁺ precursor is NMN.

A variety of different NAD pathway substrates can also be used. Exemplary suitable NAD pathway substrates include but are not limited to ethanol, lactate, glyceraldehyde-3-phosphate, malate, and glutamate. In certain embodiments, the NAD pathway substrate is selected from the group consisting of lactate, glyceraldehyde-3-phosphate, malate, glutamate, and combinations thereof.

In other embodiments, the effective amount of a NAD pathway substrate in the culture medium is from about 125 μM to about 1000 μM, alternatively from about 150 μM to about 950 μM, alternatively from about 125 μM to about 900 μM, alternatively from about 200 μM to about 800 μM, alternatively from about 250 μM to about 750 μM, alternatively from about 225 μM to about 700 μM, alternatively from about 250 μM to about 600 μM, alternatively from about 300 μM to about 600 μM, alternatively from about 350 μM to about 600 μM, alternatively from about 400 μM to about 600 μM, alternatively about 500 μM.

Similarly, a variety of different basic culture media may be used to generate the NAD⁺ precursor and/or NAD substrate containing culture media of the disclosure. In certain embodiments, the basic culture medium is BO-IVC, bEPSCM, or mTeSR.

In one embodiment, the basic culture medium is DMEM/F12 supplemented with bFGF, LiCl, GABA, pipecolic acid and TGFβ. In another embodiment, the basic culture medium is DMEM/F12 supplemented with bFGF, LiCl, TGFß, and optionally GABA, pipecolic acid, or combinations thereof. In further embodiments, the basic culture medium is supplemented with bFGF, LiCl, GABA, pipecolic acid and TGFß. In additional embodiments, the basic culture medium is supplemented with bFGF, LiCl, TGFß, and optionally GABA, pipecolic acid, or combinations thereof.

The basic culture medium can further be supplemented with each of the following: (1) XAV939 (3,5,7,8-Tetrahydro-2-[4-(trifluoromethyl)phenyl]-4H-thiopyrano[4,3-d]pyrimidin-4-one) or IWR-1 (4-[(3aR,4S,7R,7aS)-1,3-dioxo-1,3,3a,4,7,7a-hexahydro-2H-4,7-methanoisoindol-2-yl]-N-(quinolin-8-yl)benzamide; (2) CHIR99021 (6-[2-[[4-(2,4-dichlorophenyl)-5-(5-methyl-1H-imidazol-2-yl)pyrimidin-2-yl]amino]ethylamino]pyridine-3-carbonitrile); (3) WH-4-023 ((2,6-dimethylphenyl) N-(2,4-dimethoxyphenyl)-N-[2-[4-(4-methylpiperazin-1-yl)anilino]pyrimidin-4-yl]carbamate); (4) A419259 (7-[4-(4-methylpiperazin-1-yl)cyclohexyl]-5-(4-phenoxyphenyl)pyrrolo[2,3-d]pyrimidin-4-amine); (5) Vitamin C; (6) Activin A; and (7) LIF.

In an alternate embodiment, the basic culture medium is DMEM/F12 supplemented with bFGF, LiCl, GABA, pipecolic acid, TGFß, XAV939 (or IWR-1), CHIR99021, WH-4-023, A419259, Vitamin C, Activin A, and LIF. In another embodiment, the basic culture medium is DMEM/F12 supplemented with bFGF, LiCl, GABA, pipecolic acid, TGFß, XAV939, CHIR99021, WH-4-023, A419259, Vitamin C, Activin A, and LIF. In yet another embodiment, the basic culture medium is DMEM/F12 supplemented with bFGF, LiCl, GABA, pipecolic acid, TGFß, IWR-1, CHIR99021, WH-4-023, A419259, Vitamin C, Activin A, and LIF.

In an alternate embodiment, the basic culture medium is DMEM/F12 supplemented with bFGF, LiCl, TGFβ, XAV939 (or IWR-1), CHIR99021, WH-4-023, A419259, Vitamin C, Activin A, LIF, and optionally GABA, pipecolic acid, or combinations thereof. In another embodiment, the basic culture medium is DMEM/F12 supplemented with bFGF, LiCl, TGFß, XAV939, CHIR99021, WH-4-023, A419259, Vitamin C, Activin A, LIF, and optionally GABA, pipecolic acid, or combinations thereof. In yet another embodiment, the basic culture medium is DMEM/F12 supplemented with bFGF, LiCl, TGFß, IWR-1, CHIR99021, WH-4-023, A419259, Vitamin C, Activin A, LIF, and optionally GABA, pipecolic acid, or combinations thereof. In certain embodiments, the culture medium may also be supplemented with a ROCK inhibitor such as e.g., Y-27632.

In an alternate embodiment, the basic culture medium is supplemented with bFGF, LiCl, GABA, pipecolic acid, TGFβ, XAV939 (or IWR-1), CHIR99021, WH-4-023, A419259, Vitamin C, Activin A, and LIF. In another embodiment, the basic culture medium is DMEM/F12 supplemented with bFGF, LiCl, GABA, pipecolic acid, TGFß, XAV939, CHIR99021, WH-4-023, A419259, Vitamin C, Activin A, and LIF. In yet another embodiment, the basic culture medium is supplemented with bFGF, LiCl, GABA, pipecolic acid, TGFβ, IWR-1, CHIR99021, WH-4-023, A419259, Vitamin C, Activin A, and LIF.

In an alternate embodiment, the basic culture medium is supplemented with bFGF, LiCl, TGFβ, XAV939 (or IWR-1), CHIR99021, WH-4-023, A419259, Vitamin C, Activin A, LIF, and optionally GABA, pipecolic acid, or combinations thereof. In another embodiment, the basic culture medium is supplemented with bFGF, LiCl, TGFβ, XAV939, CHIR99021, WH-4-023, A419259, Vitamin C, Activin A, LIF, and optionally GABA, pipecolic acid, or combinations thereof. In yet another embodiment, the basic culture medium is supplemented with bFGF, LiCl, TGFβ, IWR-1, CHIR99021, WH-4-023, A419259, Vitamin C, Activin A, LIF, and optionally GABA, pipecolic acid, or combinations thereof. In certain embodiments, the culture medium may also be supplemented with a ROCK inhibitor such as e.g., Y-27632.

The culture media are particularly suitable for culturing mammalian embryos, such as e.g., non-primate embryos, primate embryos, mouse embryos, pig embryos, and bovine embryos.

### Methods of Improving the Cell State of Mammalian Embryos

Another aspect of the disclosure is directed to in vitro methods of improving the cell state in an embryo, which includes contacting a mammalian embryo with a culture medium supplemented with a nicotinamide adenine dinucleotide (NAD⁺) precursor and/or a NAD pathway substrate. In certain embodiments, the in vitro methods of improving the cell state in an embryo include contacting a mammalian embryo with a culture medium supplemented with a nicotinamide adenine dinucleotide (NAD⁺) precursor. In alternate embodiments, the in vitro methods of improving the cell state in an embryo include contacting a mammalian embryo with a culture medium supplemented with a NAD pathway substrate.

In certain embodiments, the improving the cell state includes increasing the presence of an expanded blastocyst, increasing the attachment rate of the embryo, and/or increasing the derivation of embryonic stem cells from the embryo. In one embodiment, the improving the cells includes increasing the presence of an expanded blastocyst. In another embodiment, the improving includes increasing the attachment rate of the embryo. In yet another embodiment, the improving includes increasing the derivation of embryonic stem cells from the embryo.

One embodiment of the disclosure is an in vitro method of increasing the presence of an expanded blastocyst. The method includes contacting a mammalian embryo with a culture medium supplemented with: a NAD⁺ precursor; or a NAD⁺ precursor and a NAD pathway substrate.

Another embodiment of the disclosure is an in vitro method of increasing the attachment rate of an embryo. The method includes contacting a mammalian embryo with a culture medium supplemented with: a NAD⁺ precursor; or a NAD⁺ precursor and a NAD pathway substrate.

Another embodiment of the disclosure is an in vitro method of increasing the derivation of pluripotent stem cells (such as e.g., embryonic stem cells) from an embryo. The method includes contacting a mammalian embryo with a culture medium supplemented with: a NAD⁺ precursor; or a NAD⁺ precursor and a NAD pathway substrate.

Yet another embodiment of the disclosure is an in vitro method of increasing the presence of an expanded blastocyst. The method includes contacting a mammalian embryo with a culture medium supplemented with: a NAD pathway substrate; or a NAD⁺ precursor and a NAD pathway substrate.

An alternate embodiment of the disclosure is an in vitro method of increasing the attachment rate of an embryo. The method includes contacting a mammalian embryo with a culture medium supplemented with: a NAD pathway substrate; or a NAD⁺ precursor and a NAD pathway substrate.

A further embodiment of the disclosure is an in vitro method of increasing the derivation of pluripotent stem cells (such as e.g., embryonic stem cells) from an embryo. The method includes contacting a mammalian embryo with a culture medium supplemented with: a NAD pathway substrate; or a NAD⁺ precursor and a NAD pathway substrate.

The contacting in any of the methods of the disclosure includes culturing the mammalian embryo for a sufficient time to improve the cell state. In one embodiment, the contacting includes culturing the mammalian embryo for about 3 to 14 days, alternative for about 4 to 13 days, alternatively for about 5 to 12 days, alternatively for about 7 to 14 days. In other embodiments, the contacting includes culturing the mammalian embryo for at least 3 days, alternatively at least 4 days, alternatively at 5 days, alternatively at least 6 days, alternatively at least 7 days. The culturing may be carried out in a controlled air atmosphere at about 38.5° C.

In some embodiments, the contacting in any of the methods of the disclosure includes culturing the mammalian embryo in the presence of a feeder layer, such as an MEF feeder layer. In other embodiments, the contacting includes culturing the mammalian embryo under feeder-free conditions (i.e., no feeder layer is present).

In certain embodiments of the disclosure, the mammalian embryos used in the disclosure are non-primate embryos. In other embodiments, the mammalian embryos used in the disclosure are primate embryos. In alternate embodiments, the mammalian embryos used in the disclosure are embryos from livestock, such as e.g., cows, sheep, and pigs, or rodents, such as e.g., mice or rats.

In certain embodiments, the embryos used in the methods can be obtained by in vitro fertilization (IVF). Alternatively, the embryos used in the methods can be obtained by parthenogenic activation (parthenogenesis). Furthermore, the embryos used in the methods can be obtained by interspecies somatic cell nuclear transfer (iSCNT), somatic cell nuclear transfer (SCNT), intracytoplasmic sperm injection, and embryonic complementation. In certain embodiments, the embryos used in the methods are generated from induced pluripotent stem cells (iPSCs).

In certain embodiments, the mammalian embryo used in the methods was obtained using parthenogenetic activation (PA). In some embodiments, the method increases the attachment rate of mammalian PA embryos.

In other embodiments, the mammalian embryo used in methods was obtained using IVF or PA. In some embodiments, the method increases the derivation of embryonic stem cells from mammalian IVF and PA embryos. In additional embodiments, the method increases the derivation of embryonic stem cells from SCN/iSCNT embryos.

A variety of different NAD⁺ precursors are suitable for use in the any of the methods of the disclosure. In one embodiment, the NAD⁺ precursor is selected from the group consisting of tryptophan, nicotinic acid (pyridine-3-carboxylic acid) (NA), nicotinamide (nicotinic acid amide), nicotinamide mononucleotide (NMN), nicotinamide riboside, and combinations thereof. In another embodiment, the NAD⁺ precursor is selected from the group consisting of nicotinic acid (pyridine-3-carboxylic acid) (NA), nicotinamide (nicotinic acid amide), nicotinamide mononucleotide (NMN), nicotinamide riboside, and combinations thereof. In an embodiment, the NAD⁺ precursor is NA, NMN, and a combination thereof. In yet another embodiment, the NAD⁺ precursor is NA. In an alternate embodiment, the NAD⁺ precursor is NMN.

A variety of different NAD pathway substrates are suitable for use in the any of the methods of the disclosure. In certain embodiments, the NAD pathway substrates include but are not limited to ethanol, lactate, glyceraldehyde-3-phosphate, malate, and glutamate. In some embodiments, the NAD pathway substrate is selected from the group consisting of lactate, glyceraldehyde-3-phosphate, malate, glutamate, and combinations thereof.

The culture medium may be supplemented with from about 125 μM to about 1000 μM, alternatively from about 200 μM to about 1000 μM, alternatively from about 250 μM to about 1000 μM, alternatively from about 150 μM to about 950 μM, alternatively from about 125 μM to about 900 μM, alternatively from about 200 μM to about 800 μM, alternatively from about 250 μM to about 750 μM, alternatively from about 225 μM to about 700 M, alternatively from about 250 μM to about 600 μM, alternatively from about 300 μM to about 600 μM, alternatively from about 350 μM to about 600 μM, alternatively from about 400 μM to about 600 μM, alternatively about 500 μM of the NAD⁺ precursor and/or NAD pathway substrate.

Alternatively, the culture medium may be supplemented with from about 125 μM to about 1000 μM, alternatively from about 200 μM to about 1000 μM, alternatively from about 250 μM to about 1000 μM, alternatively from about 150 μM to about 950 μM, alternatively from about 125 μM to about 900 μM, alternatively from about 200 μM to about 800 μM, alternatively from about 250 μM to about 750 μM, alternatively from about 225 μM to about 700 μM, alternatively from about 250 μM to about 600 μM, alternatively from about 300 μM to about 600 μM, alternatively from about 350 μM to about 600 μM, alternatively from about 400 μM to about 600 μM, alternatively about 500 μM of the NAD⁺ precursor.

Alternatively, the culture medium may be supplemented with from about 125 μM to about 1000 μM, alternatively from about 200 μM to about 1000 μM, alternatively from about 250 μM to about 1000 μM, alternatively from about 150 μM to about 950 μM, alternatively from about 125 μM to about 900 μM, alternatively from about 200 μM to about 800 μM, alternatively from about 250 μM to about 750 μM, alternatively from about 225 μM to about 700 μM, alternatively from about 250 μM to about 600 μM, alternatively from about 300 μM to about 600 μM, alternatively from about 350 μM to about 600 μM, alternatively from about 400 μM to about 600 μM, alternatively about 500 μM of the NAD pathway substrate.

In one embodiment, the culture medium is supplemented with from about 125 μM to about 1000 μM, alternatively from about 200 μM to about 1000 μM, alternatively from about 250 μM to about 1000 μM, alternatively from about 150 μM to about 950 μM, alternatively from about 125 μM to about 900 μM, alternatively from about 200 μM to about 800 μM, alternatively from about 250 μM to about 750 μM, alternatively from about 225 μM to about 700 μM, alternatively from about 250 μM to about 600 μM, alternatively from about 300 M to about 600 μM, alternatively from about 350 μM to about 600 μM, alternatively from about 400 μM to about 600 μM, alternatively about 500 μM of NA.

In another embodiment, the culture medium is supplemented with from about 125 μM to about 1000 μM, alternatively from about 200 μM to about 1000 μM, alternatively from about 250 μM to about 1000 μM, alternatively from about 150 μM to about 950 μM, alternatively from about 125 μM to about 900 μM, alternatively from about 200 μM to about 800 μM, alternatively from about 250 μM to about 750 μM, alternatively from about 225 μM to about 700 μM, alternatively from about 250 μM to about 600 μM, alternatively from about 300 μM to about 600 μM, alternatively from about 350 μM to about 600 μM, alternatively from about 400 μM to about 600 μM, alternatively about 500 μM of NMN.

In some embodiments, the contacting includes contacting the cell with an effective amount of a NAD⁺ precursor to improve state of the embryo. Such an effective amount may range from about 125 μM to about 1000 μM, alternatively from about 200 μM to about 1000 μM, alternatively from about 250 μM to about 1000 μM, alternatively from about 150 μM to about 950 μM, alternatively from about 125 μM to about 900 μM, alternatively from about 200 μM to about 800 μM, alternatively from about 250 μM to about 750 μM, alternatively from about 225 μM to about 700 μM, alternatively from about 250 μM to about 600 μM, alternatively from about 300 μM to about 600 μM, alternatively from about 350 μM to about 600 μM, alternatively from about 400 μM to about 600 μM, alternatively about 500 μM of the NAD⁺ precursor, such as e.g. NMN and/or NA.

In alternate embodiments, the contacting includes contacting the cell with an effective amount of a NAD⁺ precursor and/or a NAD pathway substrate to improve state of the embryo. Such an effective amount may range from about 125 μM to about 1000 μM, alternatively from about 200 μM to about 1000 μM, alternatively from about 250 μM to about 1000 μM, alternatively from about 150 μM to about 950 μM, alternatively from about 125 μM to about 900 μM, alternatively from about 200 μM to about 800 μM, alternatively from about 250 μM to about 750 μM, alternatively from about 225 μM to about 700 μM, alternatively from about 250 μM to about 600 μM, alternatively from about 300 μM to about 600 μM, alternatively from about 350 μM to about 600 μM, alternatively from about 400 μM to about 600 μM, alternatively about 500 μM of the NAD pathway substrate.

In certain embodiments, the contacting includes changing the culture medium. For instance, in one embodiment, the method includes culturing the mammalian embryo for at least 3 days, alternatively at least 4 days, alternatively at 5 days, which is followed by further culturing the mammalian embryo in a culture medium for at least 7 days. The further culturing may include culturing the mammalian embryo on a feeder layer. In certain embodiments, when the mammalian embryo is cultured on a feeder layer, the culture medium during the culturing is further supplemented with a rho-associated kinase (ROCK) inhibitor. For example, in certain embodiments, the further culturing includes culturing the mammalian embryo on a feeder layer in a culture medium that is supplemented with a ROCK inhibitor for the last twelve hours of culturing. In one embodiment, the ROCK inhibitor is Y27632. In other embodiments, a ROCK inhibitor is used after passaging.

In another embodiment, the method includes culturing the mammalian embryo for at least 3 days, alternatively at least 4 days, alternatively at 5 days, followed by further culturing the mammalian embryo in a culture medium for at least 7 days, which is followed by transferring the embryo to a culture medium for a further at least 7 days. The further culturing and the culturing after transferring may include culturing the mammalian embryo on a feeder layer. The further culturing and/or the culturing after transferring may include culturing the mammalian embryo on a feeder layer. In certain embodiments, when the embryo is cultured on a feeder layer, the culture medium during the culturing/and or during the culturing after the transferring is further supplemented with a ROCK inhibitor, such as e.g., Y27632. In other embodiments, the further culturing and/or the culturing after transferring include culturing the mammalian embryo on a feeder layer in a culture medium that is supplemented with a ROCK inhibitor for the last twelve hours of culturing.

In certain embodiments of the disclosure, the methods of the disclosure include further steps before and/or after the contacting a mammalian embryo with a culture medium supplemented with a NAD⁺ precursor and/or a NAD pathway substrate. For example, certain embodiments, the methods include isolating or obtaining the mammalian embryo. In other embodiments, the methods include passaging the mammalian embryo after contacting.

In certain embodiments, in which the embryo is treated with a NAD⁺ precursor, the improving the cell state is relative to an embryo not treated with the NAD⁺ precursor under otherwise identical culture conditions. Similarly, in other embodiments, the increasing the presence of an expanded blastocyst, increasing the attachment rate of the embryo, and/or increasing the derivation of embryonic stem cells from the embryo is relative to an embryo not treated with the NAD⁺ precursor under otherwise identical culture conditions.

In other embodiments, in which the embryo is treated with a NAD pathway substrate, the improving the cell state is relative to an embryo not treated with the NAD pathway under otherwise identical culture conditions. Similarly, in other embodiments, the increasing the presence of an expanded blastocyst, increasing the attachment rate of the embryo, and/or increasing the derivation of embryonic stem cells from the embryo is relative to an embryo not treated with the NAD pathway under otherwise identical culture conditions.

In certain embodiments, the disclosure is directed to in vitro methods of improving the cell state in an embryo, which includes contacting an embryo with a culture medium supplemented with a NAD⁺ precursor. The improving the cell state in these methods includes increasing the presence of an expanded blastocyst, increasing the attachment rate of the embryo, and/or increasing the derivation of embryonic stem cells from the embryo.

In other embodiments, the disclosure is directed to in vitro methods of improving the cell state in an embryo, which includes contacting an embryo with a culture medium supplemented with a NAD pathway substrate. The improving the cell state in these methods includes increasing the presence of an expanded blastocyst, increasing the attachment rate of the embryo, and/or increasing the derivation of embryonic stem cells from the embryo.

In some embodiments, the methods include culturing only embryos that are beyond the eight cell stage.

In one embodiment, the culture medium is BO-IVC, bEPSCM, or mTeSR. In one embodiment, the culture medium is DMEM/F12 supplemented with bFGF, LiCl, GABA, pipecolic acid and TGFß. In another embodiment, the culture medium is DMEM/F12 supplemented with bFGF, LiCl, TGFß, and optionally GABA, pipecolic acid, or combinations thereof. In an alternate embodiment, the culture medium is a basic medium supplemented with bFGF, LiCl, GABA, pipecolic acid and TGFß. In a further embodiment, the culture medium is basic medium supplemented with bFGF, LiCl, TGFß, and optionally GABA, pipecolic acid, or combinations thereof. The culture medium can further be supplemented with each of the following: XAV939 or IWR-1; CHIR99021; WH-4-023; A419259; Vitamin C; Activin A; and LIF.

In another embodiment, the culture medium is DMEM/F12 supplemented with bFGF, LiCl, GABA, pipecolic acid, TGFβ, XAV939 (or IWR-1), CHIR99021, WH-4-023, A419259, Vitamin C, Activin A, and LIF. In an alternate embodiment, the culture medium is DMEM/F12 supplemented with bFGF, LiCl, GABA, pipecolic acid, TGFß, XAV939, CHIR99021, WH-4-023, A419259, Vitamin C, Activin A, and LIF. In yet another embodiment, the culture medium is DMEM/F12 supplemented with bFGF, LiCl, GABA, pipecolic acid, TGFβ, IWR-1, CHIR99021, WH-4-023, A419259, Vitamin C, Activin A, and LIF.

In an alternate embodiment, the culture medium is DMEM/F12 supplemented with bFGF, LiCl, TGFβ, XAV939 (or IWR-1), CHIR99021, WH-4-023, A419259, Vitamin C, Activin A, LIF, and optionally GABA, pipecolic acid, or combinations thereof. In another embodiment, the culture medium is DMEM/F12 supplemented with bFGF, LiCl, TGFß, XAV939, CHIR99021, WH-4-023, A419259, Vitamin C, Activin A, LIF, and optionally GABA, pipecolic acid, or combinations thereof. In yet another embodiment, the culture medium is DMEM/F12 supplemented with bFGF, LiCl, TGFß, IWR-1, CHIR99021, WH-4-023, A419259, Vitamin C, Activin A, LIF, and optionally GABA, pipecolic acid, or combinations thereof.

Without further description, it is believed that one of ordinary skill in the art can, using the preceding description and the following illustrative examples, make and utilize the present invention and practice the claimed methods. The following working examples, therefore, specifically point out the preferred embodiments of the present invention and are not to be construed as limiting in any way the remainder of the disclosure.

### Examples

### Example 1: Effect of NAD⁺ Precursors on Restoring Early Embryo Development and Derivation of Embryonic Stem Cells in Mammals

Nicotinamide adenine dinucleotide (NAD⁺), a co-enzyme involved in numerous biochemical reactions, is a central hub in various biological processes. NAD⁺ is primarily synthesized in mammalian cells through NAD⁺ precursors to replenish what NADase uses during essential activities like DNA repair, metabolism, and cell death. When NAD⁺ metabolism goes awry, it is associated with several age-related diseases linked to NAD⁺ imbalances. The in vitro effect of NAD⁺ precursors on the embryonic culture of embryonic development, micromanipulated embryos/oocytes survival rate, and efficacy of in vitro Embryonic Stem Cells (ESCs) derivation are mainly unknown. However, NAD⁺ precursor metabolites are emerging as important regulators of both cell metabolism and cell state in the pluripotency state (Lees J G et al., Stem Cells 2020; 38:624-638.)

### Aim

The aim of the testing shown in this example was to evaluate the effect of NAD⁺ precursors in vitro supplementation during the early embryonic stage development (Experiment 1) and ESCs derivation (Experiment 2) in non-model species.

### Methods

The NAD⁺ precursors Nicotinic acid mononucleotide (NMN) or Nicotinic acid mononucleotide (NA) (SG; Sigma N7764) were supplemented into in vitro culture medium (BO-IVC; IVF Biosciences), and bovine embryos obtained by in vitro fertilization (IVF) or parthenogenetic activation (PA) were cultured for 7-14 days in air controlled atmosphere (5% CO₂ and 5% O₂) at 38.5° C. Embryo development was evaluated at day 3 (D3-cleavage rate), day 7 (D7−Blastocyst rate), and day 11 (D11−Hatching rate). In addition, the expansion of hatched blastocysts from day 11-13 was evaluated. Based on this testing (see in Experiment 1 below) suitable dosages of NAD⁺ precursors were identified.

After identifying the NAD⁺ precursor dosage, experiment 2 was performed. Embryos were cultured in in vitro culture medium (BO-IVC; IVF Biosciences) supplemented with NA or NMN for 7-14 days in air controlled atmosphere (5% CO₂ and 5% O₂) at 38.5° C. Embryos cultured with NAD⁺ precursor were collected at the blastocyst stage (D7) and plated in co-culture with irradiated mouse embryonic fibroblast (MEF) and bovine ESCs medium (bEPSCM) until day 25 for bovine ESCs Derivation:

Timeline bESC Derivation:
D0-D7 Incubation of IVF-produced embryos in IVC supplemented with NAD⁺ precursor for seven days.
D7 Plating of embryos for ESC derivation on medium bEPSCM on Cf1 MEF. Treatment with Y27632 for last 12 hours of culturing.
D14 Mechanically isolate the ICM and transfer to a new plate. Treatment with Y27632 for last 12 hours of culturing.
D18 Chemical isolation of bESC colonies and passage. Treatment with Y27632 for last 12 hours of culturing.
D25 Alkaline phosphatase (AP) staining and presumptive bESC/AP+ colonies counting.

### Experiment #1

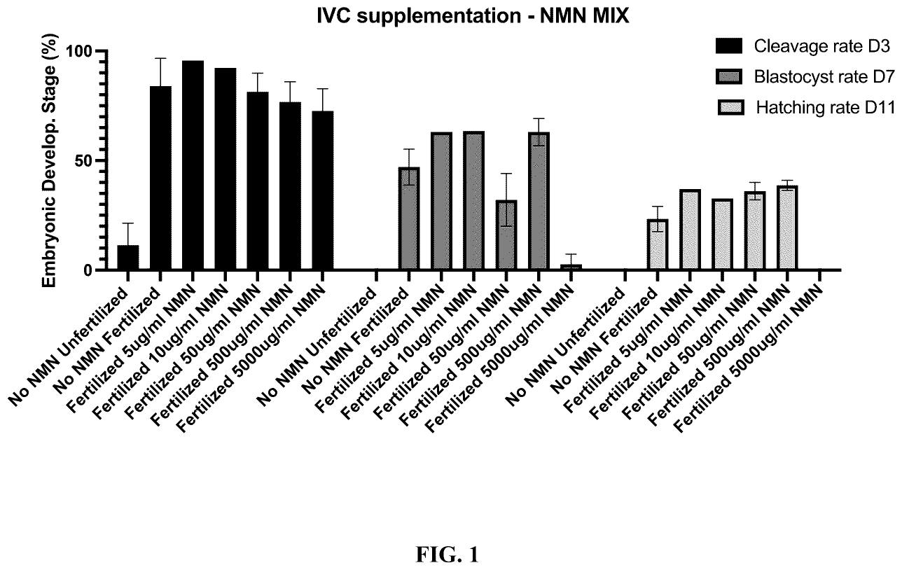

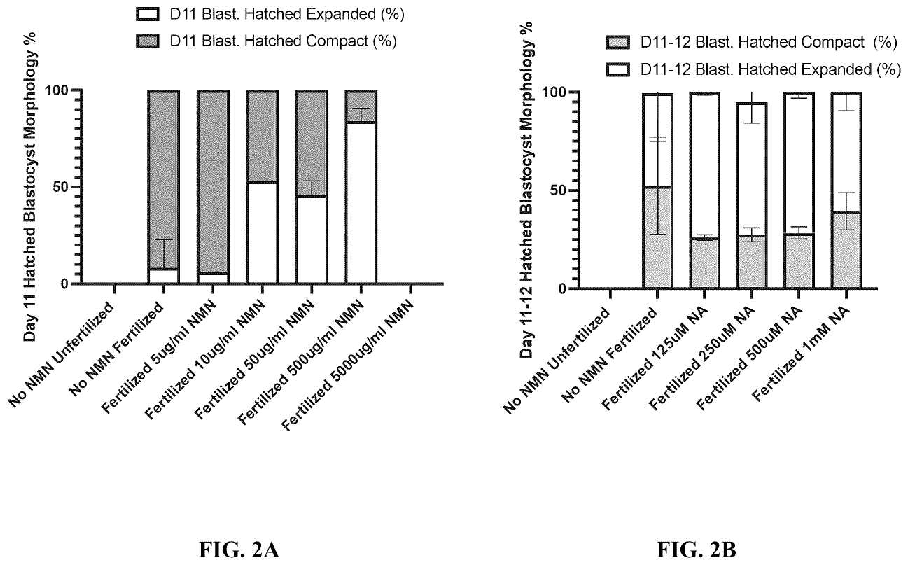

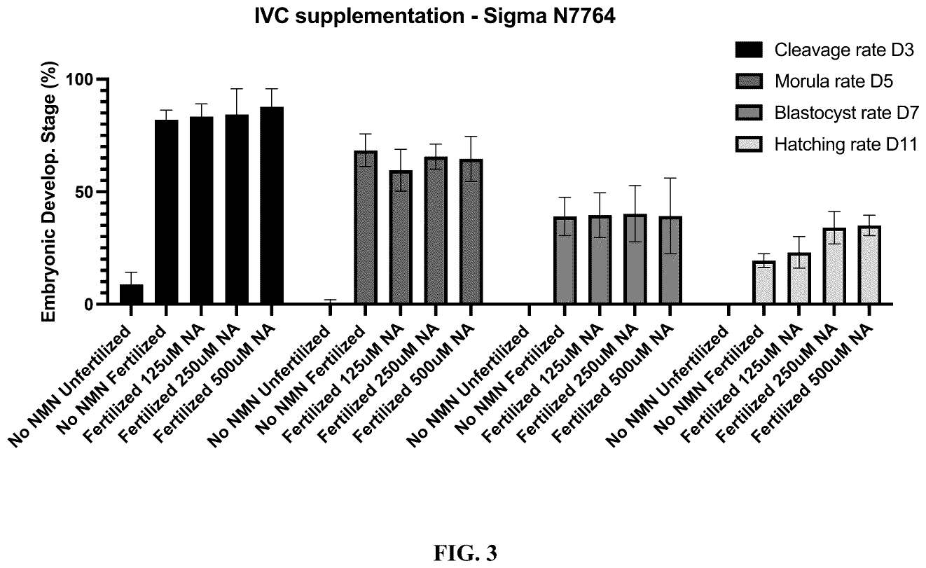

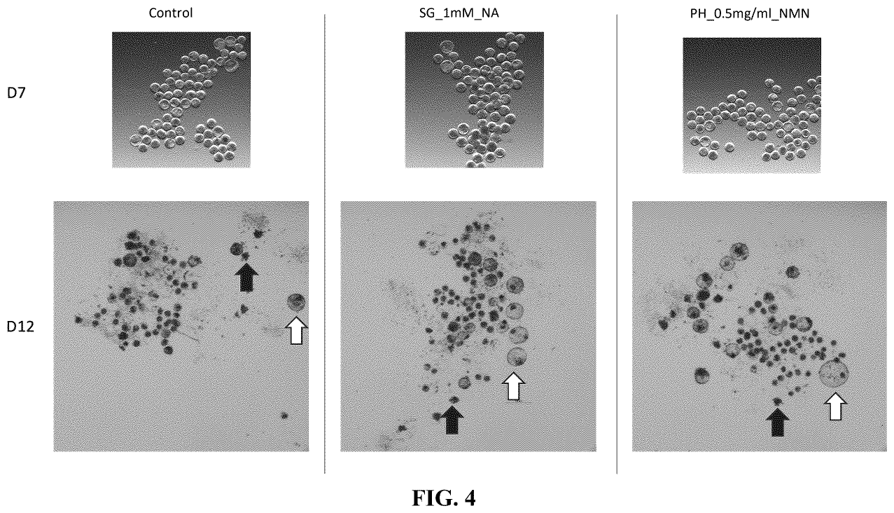

Different doses of NMN (125 to 1000 μM) were added to the IVC culture medium containing bovine embryos for up to 14 days. No effect of NAD⁺ precursor presence was observed on the cleavage or blastocyst rates for all three replicates (see FIG. 1, FIG. 2A, FIG. 2B, and FIG. 3). However, the impact of NAD⁺ precursor supplementation in IVC was observed to beneficially impact the morphology of hatched blastocysts from day 11 to day 13 (see FIG. 1, FIG. 2A, FIG. 2B, and FIG. 3). A higher presence of hatched blastocyst with expanded morphology was observed in the NAD⁺ precursor groups (FIG. 4).

### Experiment #2

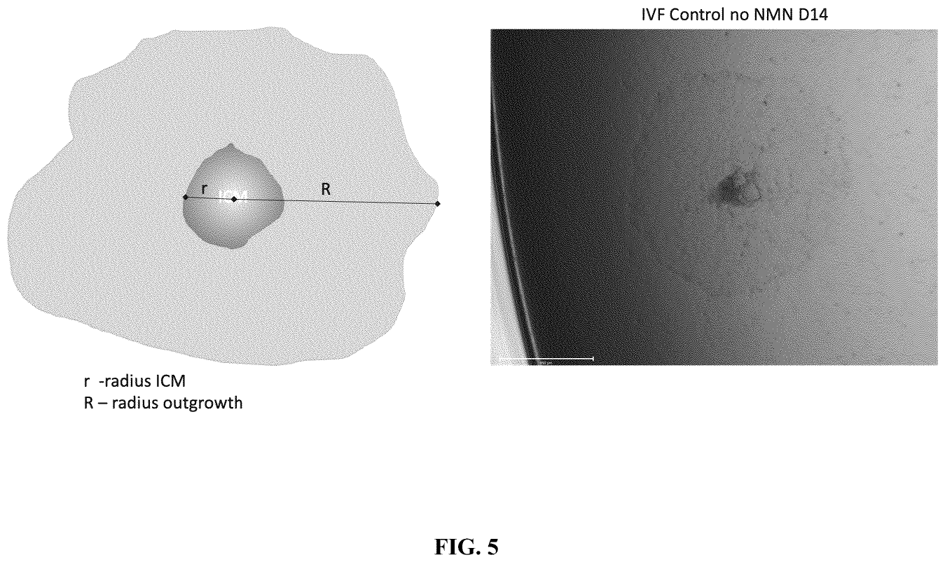

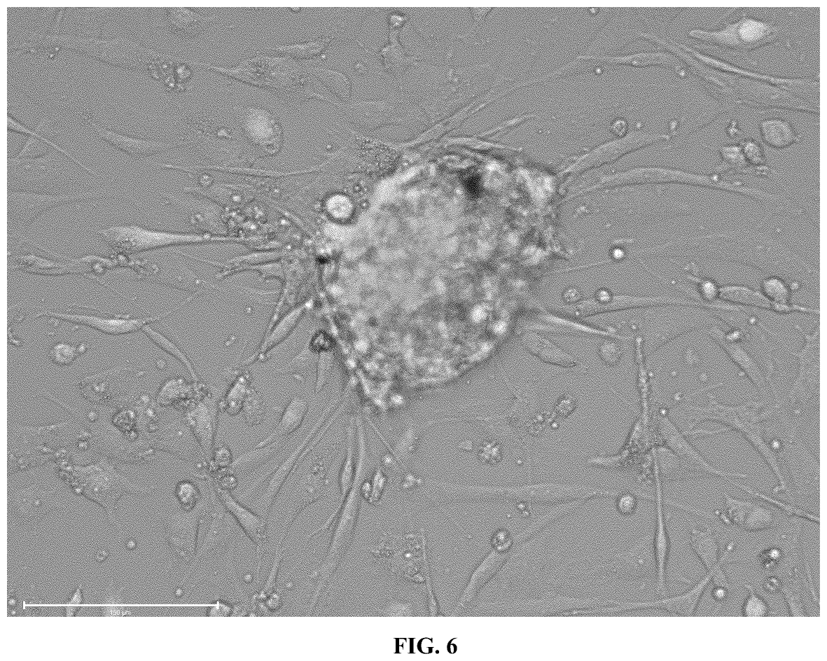

Selected doses of NAD⁺ precursors supplementation were selected based on the results from experiment 1. NMN and Nicotinic Acid (NA, also known as Niacin) were used as the NAD⁺ precursors in this experiment. On day 7 of supplementation, blastocysts were collected and plated on MEFs in a bEPSCM medium. On day 11, the attachment of the embryos was evaluated, and NAD⁺ precursor supplementation impacted the attachment rate of parthenogenetic activated (PA) embryos (Fold three times; Table 1-1). No effect was observed on in vitro fertilized embryos. On day 14, the Inner cell mass (ICM) and outgrowth radius were measured (FIG. 5, Table 1-2). No effect of treatment was observed. Seven days after the chemical passage, on day 25, the number of colonies of presumptive bESC cells was counted (FIG. 6, Table 1-3) by Alkaline Phosphatase staining (FIG. 6). Supplementation with NA and NMN during the first seven days of embryo development increased by three times the number of presumptive bESC colonies for the IVF and PA groups (Table 1-3 and Table 1-4).

TABLE 1-1
Attachment rate of embryos at day 11 of culture.

| Activation | Group | % Attached | Total # embryos | Fold vs Control |
|---|---|---|---|---|
| IVF | NA | 100% | 8 | 1.33 |
| IVF | NMN | 100% | 8 | 1.33 |
| IVF | Control | 75% | 8 | 1 |
| PA | NA | 60% | 5 | 3 |
| PA | NMN | 60% | 5 | 3 |
| PA | Control | 20% | 5 | 1 |

TABLE 1-2
Measurement of ICM and outgrowths for
day 14 embryos before ICM isolation

| | | AVG Outgrowth | AVG ICM | AVG Fold |
|---|---|---|---|---|
| Activation | Group | (μm) | (μm) | (ICM/outgrowth) |
| IVF | NA | 905.9182099 | 76.26873898 | 0.09417348 |
| n = 8 | NMN | 645.1686508 | 58.37742504 | 0.112416601 |
| | Control | 739.8561508 | 76.47707231 | 0.117377356 |
| PA | NA | 436.7380952 | 48.9047619 | 0.13030775 |
| n = 5 | NMN | 458.4821429 | 47.20238095 | 0.184470996 |
| | Control | 324.484127 | 45.91269841 | 0.157437963 |

TABLE 1-3
Average presumptive bESC colonies at day 25 of culture for in vitro fertilized embryos (IVF)

| | | | | | Number | | | |
|---|---|---|---|---|---|---|---|---|
| | | | # Seed | | of AP⁺ | # colonies | | Fold vs |
| Replicate | Activation | Group | Embryos | Area¹ | Colonies | per cm² | Average | Control |
| 1 | IVF | NA | 1 | 3.5 | 287 | 82 | 54.0952381 | 2.95833333 |
| 1 | IVF | NA | 1 | 3.5 | 253 | 72.28571429 | | |
| 1 | IVF | NA | 1 | 3.5 | 28 | 8 | | |
| 2 | IVF | NA | 5 | 3.5 | 117 | 33.42857143 | 33.42857143 | 3.25 |
| 1 | IVF | NMN | 1 | 3.5 | 39 | 11.14285714 | 12.76190476 | 0.69791667 |
| 1 | IVF | NMN | 1 | 3.5 | 57 | 16.28571429 | | |
| 1 | IVF | NMN | 1 | 3.5 | 38 | 10.85714286 | | |
| 2 | IVF | NMN | 5 | 3.5 | 57 | 16.28571429 | 16.28571429 | 1.58333333 |
| 1 | IVF | Control | 1 | 3.5 | 118 | 33.71428571 | 18.28571429 | 1 |
| 1 | IVF | Control | 1 | 3.5 | 18 | 5.142857143 | | |
| 1 | IVF | Control | 1 | 3.5 | 56 | 16 | | |
| 2 | IVF | Control | 5 | 3.5 | 36 | 10.28571429 | 10.28571429 | 1 |

¹12 well plate
Number of AP+ Colonies on Day 25

TABLE 1-4
Average of presumptive bESC colonies at day 25 of culture for parthenogenetic activated embryos (PA)

| | | | | | Number | | | |
|---|---|---|---|---|---|---|---|---|
| | | | # Seed | | of AP⁺ | # colonies | | Fold vs |
| Replicate | Activation | Group | Embryos | Areal | Colonies | per cm² | Average | Control |
| 1 | PA | NA | 5 | 3.5 | 53 | 15.14285714 | 15.14285714 | 2.94444444 |
| 1 | PA | NMN | 5 | 3.5 | 42 | 12 | 12 | 2.33333333 |
| 1 | PA | Control | 5 | 3.5 | 18 | 5.142857143 | 5.142857143 | 1 |

¹12 well plate
Number of AP+ Colonies on Day 25

While the invention has been described and illustrated herein by references to various specific materials, procedures, and examples, it is understood that the invention is not restricted to the particular combinations of material and procedures selected for that purpose. Numerous variations of such details can be implied as will be appreciated by those skilled in the art. It is intended that the specification and examples be considered as exemplary, only, with the true scope and spirit of the invention being indicated by the following claims. All references, patents, and patent applications referred to in this application are herein incorporated by reference in their entirety.

### EMBODIMENTS

The invention provides also the following non-limiting embodiments.

Embodiment 1. An in vitro method of improving the cell state in an embryo comprising contacting a mammalian embryo with a culture medium supplemented with a nicotinamide adenine dinucleotide (NAD⁺) precursor and/or a NAD pathway substrate.

Embodiment 2. The method of embodiment 1, wherein the contacting comprises culturing the mammalian embryo for 3 to 14 days.

Embodiment 3. The method of embodiment 1, wherein the contacting comprises culturing the mammalian embryo for at least 3 days.

Embodiment 4. The method of embodiment 3, wherein the method further comprises culturing the mammalian embryo in a culture medium for at least 7 days.

Embodiment 5. The method of embodiment 4, wherein the method further comprises transferring the embryo to a culture medium for a further at least 7 days.

Embodiment 6. The method of embodiments 4 or 5, wherein the method comprises culturing the mammalian embryo on a feeder layer.

Embodiment 7. The method of embodiment 6, wherein the culture medium is supplemented with a rho-associated protein kinase (ROCK) inhibitor for the last twelve hours of culturing.

Embodiment 8. The method of embodiment 1, wherein the method comprises culturing the mammalian embryo for at least the first seven days of embryo development.

Embodiment 9. The method of any one of embodiments 1-11, wherein improving the cell state comprises increasing the presence of an expanded blastocyst, increasing the attachment rate of the embryo, and/or increasing the derivation of embryonic stem cells from the embryo.

Embodiment 10. The method of embodiment 9, wherein improving the cell state comprises increasing the presence of an expanded blastocyst.

Embodiment 11. The method of embodiment 9, wherein improving the cell state comprises increasing the attachment rate of the embryo.

Embodiment 12. The method of embodiment 9, wherein improving the cell state comprises increasing the derivation of embryonic stem cells from the embryo.

Embodiment 13. The method of any one of embodiments 1-12, wherein the mammalian embryo was obtained by in vitro fertilization (IVF).

Embodiment 14. The method of any one of embodiments 1-12, wherein the mammalian embryo was obtained by parthenogenetic activation (PA).

Embodiment 15. The method of embodiment 14, wherein the method increases the attachment rate of PA embryos.

Embodiment 16. The method of embodiments 14 or 15, wherein the method increases the derivation of embryonic stem cells from IVF and PA embryos.

Embodiment 17. The method of any one of embodiments 1-12, wherein the mammalian embryo was obtained by interspecies somatic cell nuclear transfer, somatic cell nuclear transfer, intracytoplasmic sperm injection, or embryonic complementation.

Embodiment 18. The method of any one of embodiments 1-17, wherein the culture medium is supplemented with a NAD pathway substrate.

Embodiment 19. The method of embodiment 18, wherein the culture medium is supplemented with from about 125 μM to about 1000 μM, from about 250 μM to about 750 μM, or about 500 μM of the NAD pathway substrate.

Embodiment 20. The method of any one of embodiments 1-17, wherein the culture medium is supplemented with a NAD⁺ precursor.

Embodiment 21. The method of embodiment 20, wherein the culture medium is supplemented with from about 125 μM to about 1000 M of the NAD⁺ precursor.

Embodiment 22. The method of embodiment 21, wherein the culture medium is supplemented with from about 250 μM to about 750 M of the NAD⁺ precursor.

Embodiment 23. The method of embodiment 20, wherein the culture medium is supplemented with about 500 μM of the NAD⁺ precursor.

Embodiment 24. The method of any one of embodiments 20-23, wherein the NAD⁺ precursor is selected from the group consisting of tryptophan, nicotinic acid (pyridine-3-carboxylic acid) (NA), nicotinamide (nicotinic acid amide), nicotinamide mononucleotide (NMN), nicotinamide riboside, and combinations thereof.

Embodiment 25. The method of any one of embodiments 20-23, wherein the NAD⁺ precursor is NA and/or NMN.

Embodiment 26. The method of embodiment 25, wherein the NAD⁺ precursor is NMN.

Embodiment 27. A culture medium for improving the cell state in an embryo comprising: a. a basic culture medium; and b. an effective amount of a NAD⁺ precursor and/or a NAD pathway substrate to improve the state of cells in a mammalian embryo.

Embodiment 28. The culture medium of embodiment 27, wherein improving the cell state comprises increasing the presence of expanded blastocyst, increasing the attachment rate of embryos, and/or increasing the derivation of embryonic stem cells from the embryo.

Embodiment 29. The culture medium of embodiments 27 or 28, wherein the culture medium comprises a basic culture medium and an effective amount of a NAD⁺ precursor.

Embodiment 30. The culture medium of embodiment 29, wherein the culture medium comprises from about 125 M to about 1000 μM of the NAD⁺ precursor.

Embodiment 31. The culture medium of embodiments 29 or 30, wherein the NAD⁺ precursor is selected from the group consisting of tryptophan, NA, nicotinamide, NMN, nicotinamide riboside, and combinations thereof.

Embodiment 32. The culture medium of embodiments 29 or 30, wherein the NAD⁺ precursor is NMN, NA, or a combination thereof.

Embodiment 33. The culture medium of embodiments 29 or 30, wherein the culture medium comprises a basic culture medium and an effective amount of a NAD pathway substrate.

Embodiment 34. The culture medium of embodiment 33, wherein the culture medium comprises from about 125 μM to about 1000 μM of the NAD pathway substrate.

Embodiment 35. The culture medium of any one of embodiments 27-34 wherein the basic culture medium is BO-IVC, bEPSCM, or mTeSR.

Embodiment 36. The culture medium of any one of embodiments 27-34 wherein the basic culture medium is: DMEM/F12 supplemented with bFGF, LiCl, GABA, pipecolic acid and TGFß; or DMEM/F12 supplemented with bFGF, LiCl, TGFß, and optionally GABA, pipecolic acid, or combinations thereof.

Embodiment 37. The culture medium of any one of embodiments 27-34 wherein the basic culture medium is supplemented with: bFGF, LiCl, GABA, pipecolic acid and TGFß; or bFGF, LiCl, TGFß, and optionally GABA, pipecolic acid, or combinations thereof.

Embodiment 38. The culture medium of embodiments 36 or 37, wherein the basic culture medium is further supplemented with XAV939, CHIR99021, WH-4-023, A419259, Vitamin C, Activin A, and LIF.

Embodiment 39. The culture medium of embodiments 36 or 37, wherein the basic culture medium is further supplemented with IWR-1, CHIR99021, WH-4-023, A419259, Vitamin C, Activin A, and LIF.

What is claimed is:

1. An in vitro method of improving the cell state in an embryo comprising contacting a mammalian embryo with a culture medium supplemented with a nicotinamide adenine dinucleotide (NAD⁺) precursor and/or a NAD pathway substrate.

2. The method of claim 1, wherein the contacting comprises culturing the mammalian embryo for 3 to 14 days.

3. The method of claim 1, wherein the contacting comprises culturing the mammalian embryo for at least 3 days.

4. The method of claim 3, wherein the method further comprises culturing the mammalian embryo in a culture medium for at least 7 days.

5. The method of claim 4, wherein the method further comprises transferring the embryo to a culture medium for a further at least 7 days.

6. The method of claim 4, wherein the method comprises culturing the mammalian embryo on a feeder layer.

7. The method of claim 6, wherein the culture medium is supplemented with a rho-associated protein kinase (ROCK) inhibitor for the last twelve hours of culturing.

8. The method of claim 1, wherein the method comprises culturing the mammalian embryo for at least the first seven days of embryo development.

9. The method of claim 1, wherein improving the cell state comprises increasing the presence of an expanded blastocyst, increasing the attachment rate of the embryo, and/or increasing the derivation of embryonic stem cells from the embryo.

10. The method of claim 9, wherein improving the cell state comprises increasing the presence of an expanded blastocyst.

11. The method of claim 9, wherein improving the cell state comprises increasing the attachment rate of the embryo.

12. The method of claim 9, wherein improving the cell state comprises increasing the derivation of embryonic stem cells from the embryo.

13. The method of claim 1, wherein the mammalian embryo was obtained by in vitro fertilization (IVF).

14. The method of claim 13, wherein the method increases the derivation of embryonic stem cells from IVF embryos.

15. The method of claim 1, wherein the mammalian embryo was obtained by parthenogenetic activation (PA).

16. The method of claim 15, wherein the method increases the attachment rate of PA embryos and/or the derivation of embryonic stem cells from PA embryos.

17. The method of claim 1, wherein the mammalian embryo was obtained by interspecies somatic cell nuclear transfer, somatic cell nuclear transfer, intracytoplasmic sperm injection, or embryonic complementation.

18. The method of claim 1, wherein the culture medium is supplemented with a NAD pathway substrate.

19. The method of claim 18, wherein the culture medium is supplemented with from about 125 μM to about 1000 μM, from about 250 μM to about 750 μM, or about 500 μM of the NAD pathway substrate.

20. The method of claim 18, wherein the NAD pathway substrate is selected from the group consisting of lactate, glyceraldehyde-3-phosphate, malate, glutamate, and combinations thereof.

21. The method of claim 1, wherein the culture medium is supplemented with a NAD⁺ precursor.

22. The method of claim 21, wherein the culture medium is supplemented with from about 125 μM to about 1000 μM, 250 μM to about 750 μM, or about 500 μM of the NAD⁺ precursor.

23. The method of claim 21, wherein the NAD⁺ precursor is selected from the group consisting of tryptophan, nicotinic acid (pyridine-3-carboxylic acid) (NA), nicotinamide (nicotinic acid amide), nicotinamide mononucleotide (NMN), nicotinamide riboside, and combinations thereof.

24. The method of claim 21, wherein the NAD⁺ precursor is NA and/or NMN.

25. The method of claim 24, wherein the NAD⁺ precursor is NMN.

26. A culture medium for improving the cell state in an embryo comprising:
a basic culture medium; and
an effective amount of a NAD⁺ precursor and/or a NAD pathway substrate to improve the state of cells in a mammalian embryo.

27. The culture medium of claim 26, wherein improving the cell state comprises increasing the presence of an expanded blastocyst, increasing the attachment rate of the embryo, and/or increasing the derivation of embryonic stem cells from the embryo.

28. The culture medium of claim 26, wherein the culture medium comprises a basic culture medium and an effective amount of a NAD⁺ precursor.

29. The culture medium of claim 28, wherein the culture medium comprises from about 125 μM to about 1000 μM of the NAD⁺ precursor.

30. The culture medium of claim 28, wherein the NAD⁺ precursor is selected from the group consisting of tryptophan, NA, nicotinamide, NMN, nicotinamide riboside, and combinations thereof.

31. The culture medium of claim 30, wherein the NAD⁺ precursor is NMN, NA, or a combination thereof.

32. The culture medium of claim 26, wherein the culture medium comprises a basic culture medium and an effective amount of a NAD pathway substrate.

33. The culture medium of claim 32, wherein the NAD pathway substrate is selected from the group consisting of lactate, glyceraldehyde-3-phosphate, malate, glutamate, and combinations thereof.

34. The culture medium of claim 32, wherein the culture medium comprises from about 125 μM to about 1000 μM of the NAD pathway substrate.

35. The culture medium of claim 26, wherein the basic culture medium is BO-IVC, bEPSCM, or mTeSR.

36. The culture medium of claim 26, wherein the basic culture medium is:
(a) DMEM/F12 supplemented with bFGF, LiCl, GABA, pipecolic acid, and TGFß; or
(b) DMEM/F12 supplemented with bFGF, LiCl, TGFß, and optionally GABA, pipecolic acid, or combinations thereof.

37. The culture medium of claim 26, wherein the basic culture medium is supplemented with:
(a) bFGF, LiCl, GABA, pipecolic acid, and TGFß; or
(b) bFGF, LiCl, TGFß, and optionally GABA, pipecolic acid, or combinations thereof.

38. The culture medium of claim 37, wherein the basic culture medium is further supplemented with XAV939, CHIR99021, WH-4-023, A419259, Vitamin C, Activin A, and LIF.

39. The culture medium of claim 37, wherein the basic culture medium is further supplemented with IWR-1, CHIR99021, WH-4-023, A419259, Vitamin C, Activin A, and LIF.
